(19) United States
(12) Patent Application Publication
(10) Pub. No.: US 2025/0235582 A1
(43) Pub. Date: Jul. 24, 2025

(54) IMPLANT AND METHOD FOR COATING AN IMPLANT

(71) Applicant: BIO-GATE AG, Nürnberg (DE)

(72) Inventor: Martin FABRITIUS, Solothurn (CH)

(73) Assignee: BIO-GATE AG, Nürnberg (DE)

(21) Appl. No.: 18/697,960

(22) PCT Filed: Sep. 29, 2022
(86) PCT No.: PCT/EP2022/077153

§ 371 Date: Apr. 2, 2024

(30) Foreign Application Priority Data
Oct. 5, 2021 (DE) 102021125788.3

(51) Int. Cl. A61L 27/16; A61F 2/30; A61L 27/30; A61L 27/54

(52) U.S. Cl. CPC A61L 27/16; A61F 2/30771; A61F 2/3094; A61L 27/306; A61L 27/54; A61F 2002/30677; A61F 2002/30718; A61L 2300/404; A61L 2420/02; A61L 2420/08; A61L 2430/24

(57) ABSTRACT

An implant, in particular a joint implant, has ultra-high molecular weight polyethylene as the base material and a coating thereon for oxidation and/or wear protection, the coating having a biocide layer and includes a barrier layer covering the biocide layer. Compared to the uncoated base material, the coating reduces both the access of oxygen to the base material and any material escaped from the base material.

### CROSS-REFERENCE TO RELATED APPLICATIONS

This application is a National Stage Application of PCT/EP2022/077153, filed Sep. 29, 2022, which claims benefit of priority to German Patent Application No. 102021125788.3, filed Oct. 5, 2021, and which applications are incorporated herein by reference. To the extent appropriate, a claim of priority is made to each of the above disclosed applications.

### FIELD OF THE INVENTION

The invention relates to a medical technology implant and a method for coating an implant.

### PRIOR ART

EP 3 035 891 B1 discloses an anatomically adapted orthopedic implant. This is a tibial implant for implantation on a proximal tibia of a patient's knee joint. The implant has a cobalt-chromium alloy as the base material. Biocompatible plastics such as high molecular weight polyethylene (HMWPE), ultra-high molecular weight polyethylene (UHMWPE) or polyether ether ketone (PEEK) are suggested for a joint element.

DE 20 2005 005 405 U1 discloses an implant for insertion into a human or animal body, which has a bone contact surface which is at least partially covered with an osteointegrative layer, an intermediate layer being provided between the osteointegrative layer and the bone contact surface of the base body. The base body according to DE 20 2005 005 405 U1 can be made, for example, from a ceramic or a polymer, in particular PEEK or UHMWPE. The osteointegrative layer can be a porous pure titanium layer. As far as the intermediate layer is concerned, it is suggested to apply it by cold gas spraying.

EP 1 790 224 B1 discloses an antimicrobial layer material which is constructed in multiple layers, that is to say from a biocide layer and a transport control layer covering said biocide layer. The antimicrobial layer material is suitable, among other things, for coating a catheter, a contact lens, an implant, a medical nail or a dental implant.

Regarding the material UHMWPE, reference is also made to the following dissertation: “Beurteilung von vernetztem UHMWPE hinsichtlich seiner Eignung als Implantatwerkstoff für Hüftgelenkschalen [in EN: Assessment of networked UHMWPE with regard to its suitability as an implant material for hip joint shells]”, Dipl.-Ing. Ingo John, TU Berlin, Oct. 23, 2003

The dissertation focuses in particular on the possibilities of FTIR analysis (Fourier transform infrared spectroscopy). The dissertation also elaborates on studies that concern the oxidation index of gamma-irradiated UHMWPE.

### SUMMARY OF THE INVENTION

The invention is based on the object of refining medical technology implants which include UHMWPE compared to the stated prior art, particularly in terms of durability.

The configurations and advantages of the invention explained below in connection with the coating method also apply correspondingly to the device, that is to say to the medical technology implant, and vice versa.

The implant has ultra-high molecular weight polyethylene as the base material and a coating thereon, which comprises a biocide layer and a barrier layer covering said biocide layer and not necessarily being absolutely impermeable. In comparison to the uncoated base material, the coating is designed to at least inhibit the access of substances that promote corrosion, that is to say promote degradation, to the base material, in particular oxygen, and to prevent or at least reduce the release of substances from the base material. The material release mentioned could, for example, be the release of components of the base material itself or the release of degradation products.

An antimicrobial layer material according to the already mentioned patent specification EP 1 790 224 B1 is particularly suitable as a coating for the implant. As part of the coating process, a multi-layer layer consisting of a biocide layer and a barrier layer is generally applied to ultra-high molecular weight polyethylene, which fulfills a mechanical function.

The ultra-high molecular weight polyethylene (UHMWPE) can be cross-linked by beta or gamma radiation, whereby the cross-linking can occur either before or after the application of the multi-layer antimicrobial layer. Applying the coating before beta or gamma irradiation has the advantage that the base material is already protected from oxygen during the irradiation. This means that the adverse effects of radicals that could arise from irradiation and attack the workpiece surface are completely or largely prevented. In particular, the need to carry out radiation crosslinking to protect the base material in a nitrogen atmosphere can be eliminated.

The radiation-crosslinked UHMWPE serving as the base material of the implant can, for example, have a molecular weight in the range of 3 million to 7 million g/mol, a molecular weight in the range of 7 million to 10 million g/mol, or even a molecular weight of more than 10 million g/mol.

In a simplified configuration, a separate biocide-containing layer is eliminated. In this case, the base material, i.e. UHMWPE, is covered in particular by a pure polysiloxane layer. The polysiloxane layer reduces the entry of oxygen or, provided it is sufficiently thick, excludes it completely, which in any case significantly increases the service life of the product. Instead of a polysiloxane layer, another oxygen-impermeable layer, optionally supplemented by a biocide layer, can also be applied to the UHMWPE.

The coating of the implant protects the base material in particular from oxidation. At the same time, biocidal active ingredients can be released through the barrier layer. The biocidal active ingredient is, for example, selected from the group consisting of silver, copper and zinc, ions thereof and metal complexes thereof, or a mixture or alloy comprising two or more of these elements.

A particular advantage of the biocide layer is that it prevents the formation of a biofilm on the surface of the implant. Overall, the coating of the implant represents a highly effective barrier both to the inside and to the outside. Among other things, the coating prevents the ultra-high molecular weight polyethylene from becoming brittle. Ultimately, the coating dramatically increases the service life of the implant.

Thickness and porosity of the barrier layer are preferably adjusted such that the biocidal active ingredient is released from the biocide layer through the barrier layer in an antimicrobial, but not cytotoxic, amount. The biocidal active ingredient has, for example, an average grain size of 5 to 100 nm, with the average thickness of the biocide layer being 5 to 100 nm and the average thickness of the barrier layer being 5 to 500 nm.

The base material of the barrier layer can, for example, be selected from the group comprising an organic base material, in particular a plasma polymer, a sol-gel, a lacquer and a siliconized base material. Alternatively, an inorganic base material of the barrier layer can be selected, for example, from the group comprising SiO₂, SiC, a metal oxide, in particular TiO₂ Al₂O₃, and a non-biocidal metal, in particular titanium and medical stainless steel.

The multi-layer coating only partially covers the base material. Here, the multi-layer coating covers the base material, i.e. the UHMWPE, in a surface section lying outside a sliding surface of the implant, whereas the sliding surface of the same implant is formed by uncoated UHMWPE or by UHMWPE coated in another way. In addition to articulation surfaces, conical connections of the implant can also remain uncoated.

The implant can in particular be a joint implant, for example a hip or knee joint endoprosthesis or a component for elbows, ankle joints or shoulder joints. Furthermore, the multi-layer coating, which is made up of a biocide layer and a barrier layer, can be used, for example, for surgical sutures or for ligament replacements, in particular cruciate ligaments.

In any case, advantages in terms of the oxidation index and/or wear can be achieved by coating the implant. The adjustable hydrophilicity of the biocide layer is also important. In particular, the coating can lead to better wetting with water or synovial fluid compared to hydrophobic UHMWPE, which improves the wear characteristics.

It is assumed that radiation crosslinking generally improves the wear resistance of polyethylene, but at the same time carries disadvantages in terms of oxidation, i.e. aging, of the material. This target conflict is eliminated or at least reduced by the coating on the radiation-crosslinked material. The improvements achieved in terms of oxidation compared to uncoated UHMWPE are noticeable to a depth of more than 1 mm below the surface of the base material.

The coating is applied in typical processing at a temperature of approx. 40° C., which reliably avoids any thermal damage to the base material. The layer thickness is, for example, 90 nm. Depending on the process parameters, the coating can be made transparent or colored. In any case, the shelf life of the implant can be increased by the oxidation protection achieved on the surface of the implant, in particular the surface of the prosthesis. In addition, advantages can be achieved in production and logistics by not requiring packaging under inert gas, in contrast to conventional products intended for corresponding purposes.

To the extent that oxidation still occurs in the coated implant, whose coating, among other things, provides a shield against air oxygen, it is drastically delayed compared to conventional, uncoated UHMWPE implants, which significantly increases the service life of the implant.

### BRIEF DESCRIPTION OF THE DRAWINGS

An exemplary embodiment of the invention are explained in more detail below with reference to a drawing. In the figures:

FIG. 1 shows a coated component of an implant in perspective view,

FIG. 2 shows further implant components in a sectional representation,

FIG. 3 shows in a diagram the depth-dependent oxidation index of a base material of the implant in comparison with an unclaimed comparison product.

### DETAILED DESCRIPTION OF THE INVENTION

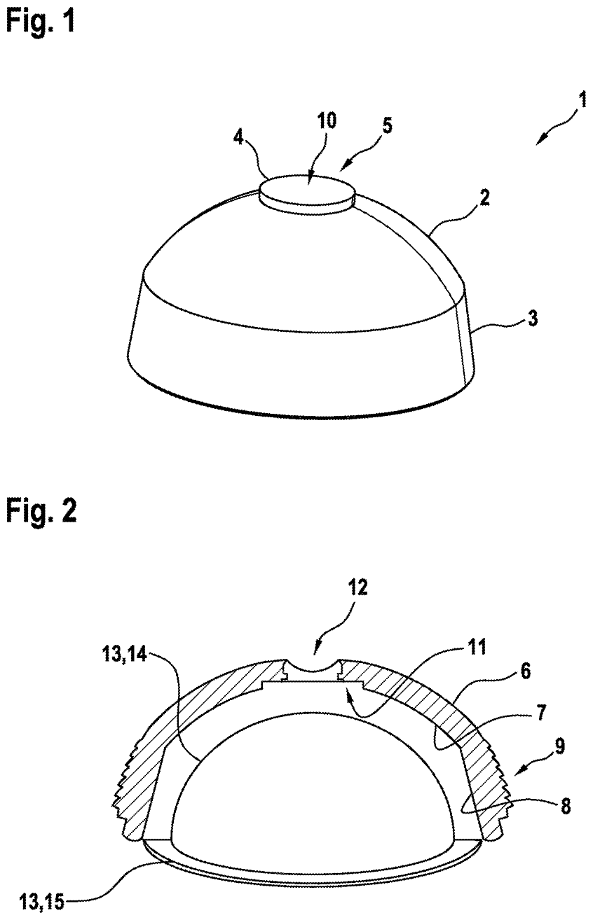

An implant, marked all together with reference numeral 1, is designed as an inlay of an artificial joint. Implant 1 has a modified hemispherical basic shape, with a spherical surface section 2 being adjoined by a frustoconical surface section 3. In the middle of spherical surface section 2, in the arrangement according to FIG. 1 at the uppermost point of implant 1, there is a disk 4, which is arranged concentrically to the central axis of the overall rotationally symmetrical implant 1 The end face of disk 4, designated 5, is provided with a coating 10, which serves to protect against wear and oxidation.

The coating 10, which is partially located on the outside of implant 1, is made up of a biocide layer and a barrier layer covering said biocide layer and is applied directly to the base material of implant 1. The base material of implant 1 is ultra-high molecular weight polyethylene, which was cross-linked using beta or gamma radiation.

Implant 1 is intended to be inserted into an outer part 6, which is visible in FIG. 2. Outer part 6, which, in contrast to implant 1, is made of a metallic material, has on its inside a concave surface section 7 and an adjoining conical section 8. The geometry of the sections 7, 8 is adapted to the shape of the spherical surface section 2 or the frustoconical surface section 3. On the outer surface of part 6, an intermeshing structure 9 can be seen in the area of conical section 8. In the middle of the inside of part 6 there is a central recess 11, which serves to insert disk 4 in a positive manner. A concavely curved recess on the outside of part 6, which is smaller in comparison to recess 11 and is also designed concentrically to the central axis of implant 1 and part 6, is designated 12. Furthermore, FIG. 2 shows an inner part 13 which interacts with implant 1 and has a spherical section 14 and an adjoining flange section 15.

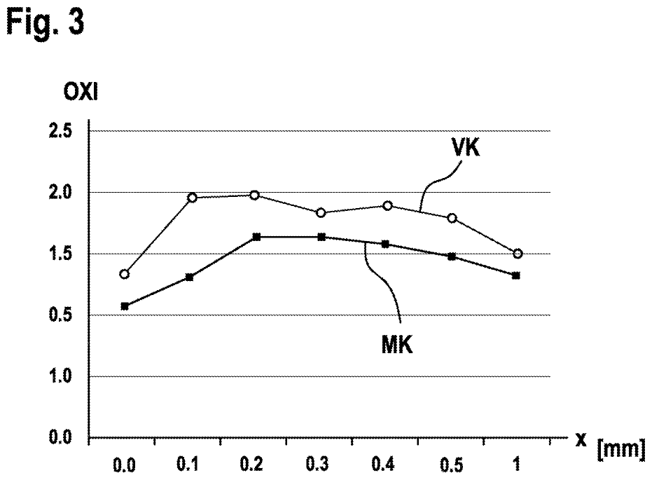

In the diagram according to FIG. 3, a measurement curve MK shows the relationship between the depth x (in millimeters) below the surface of the base material of implant 1 and the measured oxidation index OXI. The measurements were carried out on samples that were artificially aged for two weeks. The samples were coated in multiple layers, as already described in connection with FIG. 1.

In addition, a comparison curve VK is plotted in the diagram according to FIG. 3, which refers to uncoated samples, wherein—apart from the coating—the preparation of the samples and the execution of the tests were carried out in the same way as in the case of the measurement curve MK. As can be clearly seen from FIG. 3, significantly better results were achieved with the coated samples than with the uncoated comparison samples in the entire measuring range, which is reflected in the fact that the measurement curve MK is significantly below the comparison curve VK. Coating 10 thus offers effective protection against oxidation of the base material, that is, ultra-high molecular weight polyethylene.

### LIST OF REFERENCE NUMERALS

1 implant
2 spherical surface section
3 frustoconical surface section
4 disk
5 end face
6 outer part
7 concave surface section
8 conical section
9 intermeshing structure
10 coating
11 central recess
12 outside recess
13 inner part
14 spherical section
15 flange section
MK measurement curve
VK comparison curve
OXI oxidation index
x [mm] depth in mm

1. An implant, with ultra-high molecular weight polyethylene as a base material and a coating located thereon, the coating comprising a biocide layer and a barrier layer covering the base material and, compared to the uncoated base material, is adapted to reduce both access of oxygen to the base material and exit of material from the base material, wherein the coating only partially covers the base material, at a surface section lying outside a sliding surface of the implant.

2. The implant according to claim 1, wherein the biocide layer comprises a biocidal active ingredient which is selected from the group consisting of silver, copper and zinc, ions thereof and metal complexes thereof, or a mixture or alloy comprising two or more of these elements.

3. The implant according to claim 2, wherein the barrier layer has a thickness and porosity adjusted to release the biocidal active ingredient from the biocide layer through the barrier layer in an antimicrobial, non-cytotoxic amount.

4. The implant according to claim 1, wherein the biocidal active ingredient has an average grain size of 5 to 100 nm, an average thickness of the biocide layer is 5 to 100 nm, and an average thickness of the barrier layer is 5 to is 500 nm.

5. The implant according to claim 1, wherein the barrier layer has a base material which is selected from the group comprising an organic base material, a sol-gel, a lacquer and a siliconized base material.

6. The implant according to claim 1, wherein the barrier layer has an inorganic base material which is selected from the group comprising SiO₂, SiC, a metal oxide, and a non-biocidal metal.

7. A method for coating an implant, wherein a multi-layer coating comprising a biocide layer and a barrier layer is applied to ultra-high molecular weight polyethylene so that the coating only partially covers base material, at a surface section lying outside a sliding surface of the implant.

8. The method according to claim 7, wherein an oxygen-impermeable layer is applied as the barrier layer.

9. The method according to claim 7, wherein the polyethylene is cross-linked by beta or gamma radiation.

10. The method according to claim 9, wherein the polyethylene is cross-linked by radiation before the multi-layer coating is applied.

11. The method according to claim 9, wherein the polyethylene is cross-linked by radiation after the multi-layer coating is applied.

12. The implant according to claim 1, wherein the barrier layer has a base material which is selected from the group comprising a plasma polymer, a sol-gel, a lacquer and a siliconized base material.

13. The implant according to claim 1, wherein the barrier layer has an inorganic base material which is selected from the group comprising SiO₂, SiC, TiO₂, Al₂O₃, and titanium and medical grade stainless steel.
